(19) United States
(12) Patent Application Publication
(10) Pub. No.: US 2020/0096313 A1
(43) Pub. Date: Mar. 26, 2020

(54) IMAGING APPARATUS AND CONTROLLING METHOD THEREOF

(71) Applicant: SCREEN HOLDINGS CO., LTD., Kyoto (JP)

(72) Inventor: Keisuke Kato, Kyoto (JP)

(21) Appl. No.: 16/531,997

(22) Filed: Aug. 5, 2019

(30) Foreign Application Priority Data
Sep. 21, 2018 (JP) 2018-177813
May 23, 2019 (JP) 2019-096457

(51) Int. Cl. G01B 9/02; G02B 21/36

(52) U.S. Cl. CPC G01B 9/02091; A61B 3/102; G02B 21/361; G02B 21/365

(57) ABSTRACT

An imaging apparatus for optical coherence tomographic imaging of the invention includes an objective optical system which causes illumination light to be incident on an imaging object in a specimen container and receives reflected light from the imaging object, a focus adjustor which adjusts a focus position of the objective optical system in an optical axis direction with respect to the specimen container, a temperature detector which detects a temperature of the objective optical system, and a calculator which calculates an adjustment amount of the focus position for an adjustment operation by the focus adjustor based on the temperature detected by the temperature detector and a correlation relationship of the temperature and the adjustment amount obtained in advance.

### CROSS REFERENCE TO RELATED APPLICATION

The disclosure of Japanese Patent Applications enumerated below including specifications, drawings and claims is incorporated herein by reference in its entirety:

No.2018-177813 filed on Sep. 21, 2018; and

No.2019-096457 filed on May 23, 2019.

### BACKGROUND OF THE INVENTION

### 1. Field of the Invention

This invention relates to an imaging apparatus for imaging an imaging object in a specimen container by an optical coherence tomographic imaging method and a control method for the imaging apparatus.

### 2. Description of the Related Art

In technical fields of medicine and biochemistry, samples such as cells and microorganisms cultured in a container are observed. Techniques for imaging cells and the like using a microscope or the like are proposed as methods for observation without affecting the cells and the like to be observed. One of such techniques utilizes an optical coherence tomography technique. In this technique, low-coherence light emitted from a light source is caused to be incident as illumination light on an imaging object and interference light of reflected light (signal light) from the imaging object and reference light having a known optical path length is detected, whereby an intensity distribution in a depth direction of the reflected light from the imaging object is obtained for tomographic imaging.

A conventional OCT imaging technique has been often used for the purpose of observing a part of a living body such as a retina. However, the use of this technique in imaging an imaging object contained in a specimen container such as tissues obtained from a living body or cultured cells is on the increase. The applicant of this application also previously disclosed a technique suitable in imaging an imaging object carried in a specimen container such as a well plate (for example, JP 2018-105683A).

An application to so-called time lapse imaging for observing a change of the same specimen by imaging the specimen at a predetermined time interval for a long time is expected as the use of the OCT technique in imaging a specimen in a container in this way. Time lapse imaging using an optical microscope has been widely performed and it is known that a temperature change associated with the lapse of time brings about a variation of a focus position of an optical system. Further, a technique for suppressing the influence of such a focus position variation has also been proposed (for example, JP 2005-107302A).

In the OCT imaging technique, a tomographic image in a cross-section parallel to an optical axis direction is captured by calculation using interference light of signal light and reference light. In this technique, set states of an optical system such as a focus position and a reference optical path length during imaging affect the quality of an image, but how these are set is difficult to judge from the image. On the other hand, these settings are affected by a temperature during imaging. Thus, in order to apply the OCT imaging technique to time lapse imaging, variations of imaging conditions associated with a temperature change need to be suppressed. However, a conventional microscope imaging technique could not be applied to the OCT imaging having a different imaging principle.

### SUMMARY OF THE INVENTION

This invention was developed in view of the above problem and an object thereof is to provide a technique capable of suppressing an image quality variation due to a temperature change in an imaging apparatus for imaging an imaging object in a specimen container by an optical coherence tomographic imaging method.

To achieve the above object, one aspect of this invention is directed to an imaging apparatus for optical coherence tomographic imaging with an objective optical system which causes illumination light to be incident on an imaging object in a specimen container and receives reflected light from the imaging object, a focus adjustor which adjusts a focus position of the objective optical system in an optical axis direction with respect to the specimen container, a temperature detector which detects a temperature of the objective optical system, and a calculator which calculates an adjustment amount of the focus position for an adjustment operation by the focus adjustor based on the temperature detected by the temperature detector and a correlation relationship of the temperature and the adjustment amount obtained in advance.

Further, to achieve the above object, another aspect of this invention is directed to a control method for optical coherence tomographic imaging apparatus, the control method including detecting a temperature of an objective optical system which causes illumination light to be incident on an imaging object in a specimen container and receives reflected light from the imaging object, calculating an adjustment amount of a focus position based on the temperature and a correlation relationship of the temperature and the adjustment amount obtained in advance, and adjusting the focus position of the objective optical system in an optical axis direction with respect to the specimen container according to the adjustment amount.

In the invention thus configured, the focus position during imaging is set according to the temperature detection result at that time based on the correlation relationship of the temperature of the objective optical system and the adjustment amount of the focus position obtained in advance. Thus, a variation of the focus position associated with a temperature change can be suppressed and an image quality variation due to this variation can be suppressed.

Further, to achieve the above object, still another aspect of this invention is directed to an imaging apparatus for optical coherence tomographic imaging with an objective optical system which causes illumination light to be incident on an imaging object in a specimen container and receives reflected light from the imaging object, a reference optical system which forms a reference optical path of reference light for the reflected light. an optical path adjustor which adjusts an optical path length of the reference optical path, a temperature detector which detects a temperature of the reference optical system, and a calculator which calculates an adjustment amount of the optical path length for an adjustment operation by the optical path adjustor based on the temperature detected by the temperature detector and a correlation relationship of the temperature and the adjustment amount obtained in advance.

Further, to achieve the above object, further another aspect of this invention is directed to a control method for optical coherence tomographic imaging apparatus, the control method including detecting a temperature of a reference optical system which forms an optical path of reference light, calculating an adjustment amount of the optical path length of the reference light based on the temperature and a correlation relationship of the temperature and the adjustment amount obtained in advance, and adjusting the optical path length according to the adjustment amount.

In the invention thus configured, the reference optical path length during imaging is set according to the temperature detection result based on the correlation relationship of the temperature of the reference optical system and the adjustment amount of the reference optical path length obtained in advance. Thus, a variation of the reference optical path length associated with a temperature change can be suppressed and an image quality variation due to this variation can be suppressed.

As described above, according to the invention, an image quality variation due to a temperature change can be suppressed by suppressing a variation of the focus position of the objective optical system or the reference optical path length of the reference optical system associated with the temperature change.

The above and further objects and novel features of the invention will more fully appear from the following detailed description when the same is read in connection with the accompanying drawing. It is to be expressly understood, however, that the drawing is for purpose of illustration only and is not intended as a definition of the limits of the invention.

### BRIEF DESCRIPTION OF THE DRAWINGS

FIG. 1 is a diagram showing a schematic configuration of one embodiment of the imaging apparatus according to the invention.

FIGS. 2A and 2B are graphs schematically showing a relationship of the temperature of the objective optical system and the focus position.

FIGS. 3A to 3C are principle diagrams showing how to obtain a focus adjustment amount corresponding to a temperature change.

FIG. 4 is a flow chart showing a process of obtaining the correlation relationship of the temperature and the focus adjustment amount.

FIG. 5 is a flow chart showing a process of obtaining a correlation relationship of the temperature and the mirror adjustment amount.

FIG. 6 is a flow chart showing time lapse imaging.

FIGS. 7A and 7B are graphs showing a method for obtaining the focus adjustment amount from the temperature characteristic of the objective optical system.

FIG. 8 is a diagram showing a modification of the imaging unit.

### DETAILED DESCRIPTION OF THE PREFERRED EMBODIMENTS

Hereinafter, a specific embodiment of an imaging apparatus according to the invention is described. In this embodiment, a specimen such as cells or cell colonies continuously cultured in an incubator is tomographically imaged by an OCT (Optical Coherence Tomography) technique. Further, the imaging apparatus has a function of generating a tomographic image and a three-dimensional image of the specimen based on data obtained by imaging. This imaging apparatus is suitable for so-called time lapse imaging for imaging a specimen cultured in a constant culture environment at a predetermined time interval.

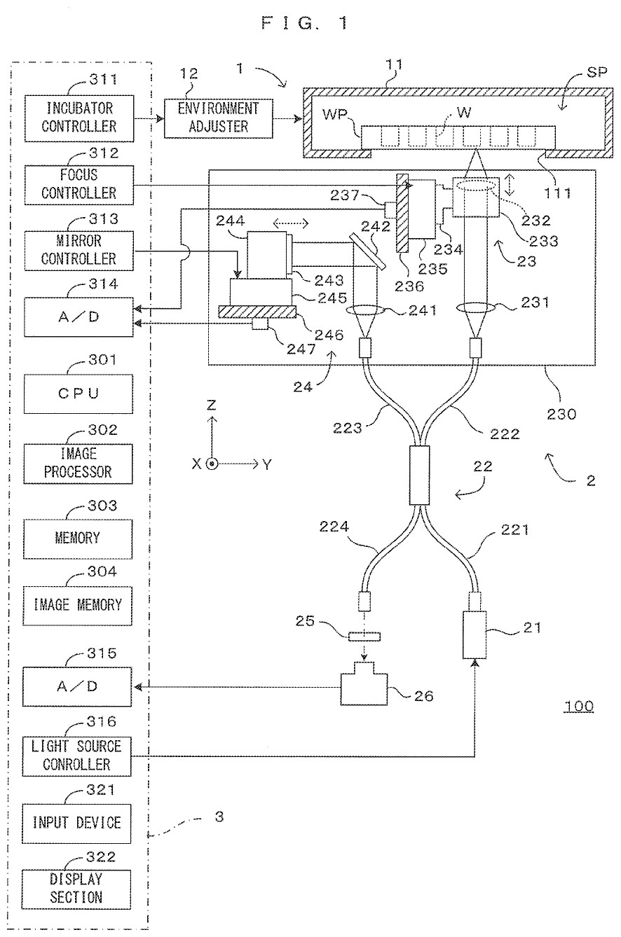

FIG. 1 is a diagram showing a schematic configuration of one embodiment of the imaging apparatus according to the invention. The configuration of this imaging apparatus 100 can be roughly divided into an incubator unit 1 for realizing a predetermined culture environment and culturing a specimen, an imaging unit 2 for OCT-imaging the specimen and a control unit 3 for controlling the incubator unit 1 and the imaging unit 2 and performing an image processing based on imaging data. To uniformly indicate directions in the drawings to be described below, XYZ orthogonal coordinate axes are set as shown in FIG. 1. Here, an XY plane represents a horizontal plane. Further, a Z axis represents a vertical axis and, more particularly, a (−Z) direction represents a vertically downward direction.

The incubator unit 1 includes a chamber 11 and an environment adjuster 12. The chamber 11 includes an internal space SP capable of accommodating a container for culturing the specimen, e.g. a well plate WP. The environment adjuster 12 adjusts temperature, humidity and an internal atmosphere of the internal space SP of the chamber 11. The chamber 11 is fixed to an unillustrated body frame of the imaging apparatus 100. The environment adjuster 12 is controlled by an incubator controller 311 provided in the control unit 3 to adjust the temperature, the humidity and the internal atmosphere in the chamber 11 to adapt to a predetermined culture environment.

A well plate (also called a microplate, a microtiter plate or the like) provided with a plurality of, e.g. 96 (=12×8) wells W in an upper surface can be suitably used as the container for the specimen. Note that the arrangement and number of the wells W are not limited to those described above and are arbitrary. Further, a container called a dish and in the form of a shallow dish can also be used. Here, it is assumed that the well plate WP having a known outer size is used.

The well plate WP is carried into the chamber 11 from outside and carried out to outside from the chamber 11 via an unillustrated opening/closing door. An opening 111 having a planar size slightly smaller than the well plate WP is provided in the bottom surface of the chamber 11. The well plate WP carried into the chamber 11 is so disposed in the chamber 11 that the bottom surface thereof faces the opening 111. The chamber 11 may be further provided with a seal member for maintaining a space between the bottom surface of the well plate WP and the bottom surface of the chamber 11 in an airtight manner, a fixing member for fixing the well plate WP to a bottom part of the chamber 11 and the like.

The specimen containing cells or the like as an imaging object is already prepared in at least some of the wells W provided in the well plate WP. The specimen is cultured for a predetermined time in the chamber 11 having the culture environment regulated, that is, constant temperature and constant humidity environment. Based on an imaging schedule prepared in advance, the imaging unit 2 operates to image the specimen every time a determined imaging time arrives. During a culturing and imaging, the well plate WP holding the specimen is kept stationary in the chamber 11. Thus, for example, displacements of the cells and the like possibly occurring due to the conveyance of the well plate WP for each imaging can be prevented.

The imaging unit 2 is arranged below the well plate WP having the bottom surface exposed from the chamber 11. An OCT (Optical Coherence tomography) device capable of capturing a tomographic image of an imaging object in a non-contact and non-destructive (non-invasive) manner is used in the imaging unit 2. Although described in detail later, the imaging unit 2, which is an OCT device, includes a light source 21 for generating illumination light to the imaging object, an optical fiber coupler 22, an objective optical system 23, a reference optical system 24, a spectrometer 25 and a photodetector 26.

In the imaging unit 2, from the light source 21 which includes a light emitting element such as a light emitting diode or a super luminescent diode (SLD) for instance, a low-coherence light beam containing wide-range wavelength components is emitted. The light source 21 is controlled by a light source controller disposed to the control unit 3. For imaging the specimen such as cells or the like, an infrared light can be used favorably to make illumination light penetrate into the specimen.

The light source 21 is connected one optical fiber 221 of optical fibers constituting the optical fiber coupler 22. Low-coherence light emitted from the light source 21 is branched into lights to two optical fibers 222, 223 by the optical fiber coupler 22. The optical fiber 222 constitutes an object side optical path. More specifically, light emitted from an end part of the optical fiber 222 is incident on an objective optical system 23.

The objective optical system 23 includes a collimator lens 231 and an objective lens 232. Light emitted from an end part of the optical fiber 222 is incident on the objective lens 232 via the collimator lens 231. The objective lens 232 has a function of converging light (illumination light) from the light source 21 to the specimen and a function of condensing reflected light from the specimen and causing the condensed reflected light toward the optical fiber coupler 22. Although a single objective lens 232 is shown in FIG. 1, a plurality of optical elements may be combined. Reflected light from the imaging object is incident as signal light on the optical fiber 222 via the objective lens 232 and the collimator lens 231. An optical axis of the objective lens 232 is orthogonal to the bottom surface of the well plate WP and, in this example, an optical axis direction coincides with a vertical axis direction.

The objective lens 232 is housed in a lens barrel 233. The lens barrel 233 is supported by an elevating member 234. Further, the elevating member 234 is supported by an elevating mechanism 235. The elevating mechanism 235 includes an appropriate mechanism for raising and lowering the elevating member 234, e.g. a linear motor or a ball screw mechanism. The elevating mechanism 235 is fixed to a supporting member 236. The supporting member 236 is a member made of metal such as stainless steel, aluminum or steel, and fixed to a base plate 230. The base plate 230 is fixed to an unillustrated body frame of the imaging apparatus 100.

The elevating mechanism 235 operates in response to a drive signal from a focus controller 312 provided in the control unit 3 to raise and lower the lens barrel 233. This causes the objective lens 232 to move along the vertical direction (Z direction), i.e. in directions toward and away from the bottom surface of the well plate WP. In this way, a focus position of the objective lens 232 in the Z direction is adjusted.

Part of light incident on the optical fiber coupler 22 from the light source 21 is incident on the reference optical system 24 via an optical fiber 223. The reference optical system 24 includes a collimator lens 241, a folding mirror 242 and a reference mirror 243. These constitute a reference system optical path together with the optical fiber 223. Specifically, light emitted from an end part of the optical fiber 223 is incident on the reference mirror 243 via the collimator lens 241 and the folding mirror 242. The light reflected by the reference mirror 243 is incident as reference light on the optical fiber 223. The folding mirror 242 contributes to the miniaturization of the apparatus by appropriately folding an optical path of the reference light.

The reference mirror 243 is mounted on a side surface of an advancing/retracting member 244, and the advancing/retracting member 244 is supported by an advancing/retracting mechanism 245. The advancing/retracting mechanism 245 includes an appropriate mechanism for advancing and retracting the advancing/retracting member 244 in a Y direction, e.g. a linear motor or a ball screw mechanism. The advancing/retracting mechanism 245 is fixed to a supporting member 246. The supporting member 246 is a member made of metal such as stainless steel, aluminum or steel, and fixed to the base plate 230.

The advancing/retracting mechanism 245 operates in response to a drive signal from a mirror controller 313 provided in the control unit 3 to advance or retract the advancing/retracting member 244 in the Y direction. This causes the reference mirror 243 to move in the Y direction, i.e. move in directions toward and away from the folding mirror 242. In this way, an optical path length of the reference light reflected by the reference mirror 243 is adjusted.

The reflected light (signal light) reflected by a surface or an internal reflecting surface of the specimen and reference light reflected by the reference mirror 243 are mixed in the optical fiber coupler 22 and incident on the photo-detector 26 via the optical fiber coupler 22. At this time, interference due to a phase difference between the reflected light and the reference light occurs, but an optical spectrum of interference light differs depending on a depth of the reflecting surface. That is, the optical spectrum of the interference light has information on a depth direction of the imaging object. Thus, a reflected light intensity distribution in the depth direction of the imaging object can be obtained by spectrally diffracting the interference light at each wavelength to detect a light quantity and Fourier transforming a detected interference signal. An OCT imaging technique based on such a principle is called Fourier domain OCT (FD-OCT).

The imaging unit 2 of this embodiment is provided with a spectroscope 25 on an optical path of the interference light from the optical fiber 224 to the photo-detector 26. A spectroscope utilizing a prism, a spectroscope utilizing a diffraction grating and the like can be, for example, used as the spectroscope 25. The interference light is spectrally diffracted for each wavelength component and received by the photo-detector 26.

By Fourier-transforming the interference signal output from the photo-detector 26 according to the interference light detected by the photo-detector 26, the reflected light intensity distribution of the specimen in the depth direction, i.e. in the Z direction at the incident position of the illumination light is obtained. By scanning the illumination light incident on the well plate WP in the X direction, the reflected light intensity distribution in a plane parallel to an XZ plane is obtained, with the result that a tomographic image of the specimen having this plane as a cross-section can be generated. A principle of generation of the tomographic image is not described because it is known.

Images are obtained by changing the incident position of the light along the Y direction over multiple steps and imaging a tomographic image for every change. By doing so, a number of tomographic images of the specimen are obtained along cross-sectional surfaces which are parallel to the XZ plane. As the scan pitch in the Y direction is reduced, it is possible to obtain image data with sufficient resolution to grasp the stereoscopic structure of the specimen. Scan movements of the light beam in X and Y direction are realized as an optical device (not shown) changing an optical path such as a Galvanometer mirror changes the incident position of the light beam to X and Y direction, the well plate WP carrying the specimen and imaging unit 2 relatively move to X and Y direction or the like.

The control unit 3 comprises a CPU (Central Processing Unit) 301, an image processor 302, a memory 303, an image memory 304 and an A/D convertor 315. The CPU 31 governs operations of the entire apparatus by executing a predetermined control program, thereby realizes various processing described later. The control program executed by the CPU 301 and data which are generated during processing are stored in the memory 303. The A/D convertor 315 converts a signal which the photo-detector 26 of the imaging unit 2 outputs in accordance with the amount of received light into digital image data. The image processor 302 performs image processing described later based upon a digital data outputted from the A/D converter 315, thereby generates a tomographic image and 3D image of the imaging object. The image memory 304 saves the image data transferred from the imaging unit 2 via the A/D converter 315 and the image data of the tomographic images and 3D images generated by the image processor 302.

Further, the control unit 3 has a user interface function for accepting manipulation by a user and informing the user of various types of information. For this purpose, an input device 321 and a display section 322 are disposed to the control unit 3. The input device 321 is for instance a key board, a mouse, a touch panel or the like which can accept manipulation and entry concerning selection of the functions of the apparatus, setting of operating conditions, etc. The display section 322 comprises a liquid crystal display for example which shows various types of processing results such as the tomographic images and the 3D images generated by the image processor 302.

In the objective optical system 23, a focus position may vary due to an ambient temperature change since the optical elements such as the objective lens 232 and the members supporting those optical elements are thermally deformed. Further, a reference optical path length may similarly vary due to an ambient temperature change also in the reference optical system 24. A variation of the focus position causes a variation of an in-focus position in a captured image. Further, a variation of the reference optical path length causes a variation of the position of the imaging object appearing in the image.

Such variations are thought not to be a big problem in single imaging. However, this should be avoided in time lapse imaging for observing a change of the same specimen by imaging the specimen at a time interval a plurality of number of times and comparing images. This is because different imaging conditions for each image make the comparison of the images difficult.

For this purpose, it is also considered to place the objective optical system 23 and the reference optical system 24 in a constant temperature environment. However, this cannot be said to be realistic if the complication of an apparatus configuration, the consumption of much energy also during a waiting time and the like are considered. Accordingly, in this embodiment, an ambient temperature of at least one of the objective optical system 23 and the reference optical system 24 during imaging is detected and at least one of the focus position and the reference mirror position is adjusted according to the detection result. By doing so, variations of imaging conditions due to a temperature change are suppressed.

Specifications required for the imaging apparatus 100 for the purpose of satisfactorily performing time lapse imaging are such that imaging conditions adjusted by a user at the time of first imaging are similarly reproduced also in imaging after a while. When a first image is captured, various imaging conditions are thought to be tried and tested by the user to obtain a desired image. The imaging conditions thus determined are required to be unchanged also in later imaging. Note that the imaging conditions mentioned here mean the focus position of the objective lens 232 in the objective optical system 23 and the reference optical path length in the reference optical system 24.

The imaging unit 2 of this embodiment is provided with a temperature sensor 237 for detecting an ambient temperature of the objective optical system 23 and a temperature sensor 247 for detecting an ambient temperature of the reference optical system 24. More specifically, the temperature sensor 237 is mounted, for example, on the supporting member 236, which is a relatively large metal member provided in the objective optical system 23. On the other hand, the temperature sensor 247 is mounted, for example, on the supporting member 246, which is a relatively large metal member provided in the reference optical system 24. The temperatures of these members are thought to typically represent the ambient temperatures of the respective objective optical system 23 and reference optical system 24.

The temperature sensors 237, 247 are desirably capable of accurately detecting a temperature around a room temperature and, for example, thermocouples, resistance temperature detectors, semiconductors and the like can be used as such. Outputs of the temperature sensors 237, 247 are converted into digital data by an A/D converter 314 of the control unit 3. The data is processed by a CPU 301 and utilized in adjusting the focus position and the reference optical path length.

The principle of adjusting the focus position based on a detection result of the ambient temperature and a specific operation are described for the objective optical system 23 below. However, a similar way of thinking can be applied also to the reference optical system 24 and common parts may not be described.

A control command including an adjustment amount for adjusting the focus position is given from the CPU 301 to the focus controller 312. The adjustment amount is, for example, a drive pulse number of a pulse motor provided as a drive source in the elevating mechanism 235. The focus controller 312 changes the focus position by operating the elevating mechanism 235 according to the given adjustment amount to move the objective lens 232. However, an actual focus position at that time is affected by the ambient temperature and not necessarily properly set at a desired position. That is, the adjustment amount output from the CPU 301 is merely for indirectly specifying the focus position. The actual focus position is determined by the adjustment amount and the ambient temperature.

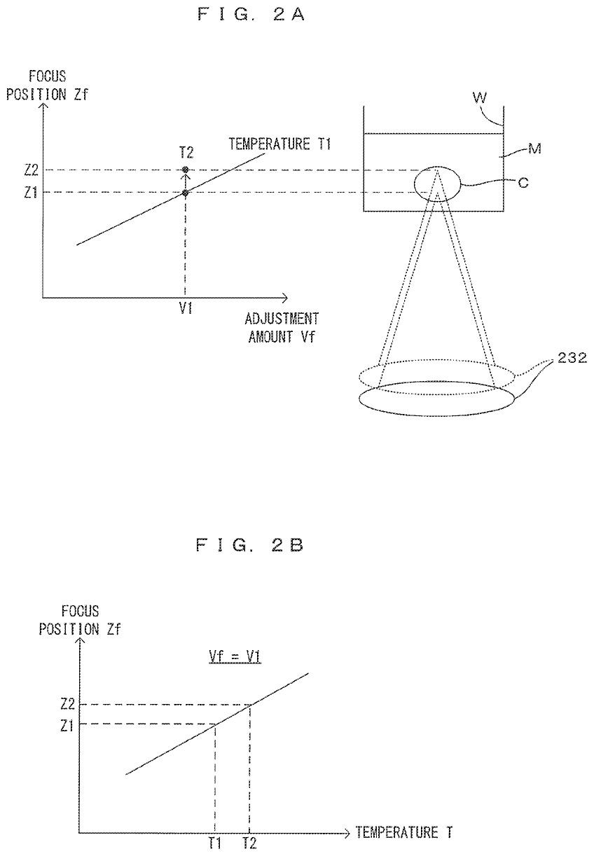

FIGS. 2A and 2B are graphs schematically showing a relationship of the temperature of the objective optical system and the focus position. For the focus position of the objective optical system 23, a control command including a focus position adjustment amount (hereinafter, referred to as a “focus adjustment amount”) Vf is given from the CPU 301 to the focus controller 312. As shown in a graph on the left side of FIG. 2A, a focus position Zf is thought to change substantially in proportion to a change of the focus adjustment amount Vf if the temperature around the objective optical system 23 is constant. For example, if T1 denotes the temperature at this time, the focus position Zf corresponding to the focus adjustment amount Vf=V1 is Z1. In FIG. 2A, M denotes a culture medium injected into the well W and C denotes a cell carried in the culture medium.

Here, it is assumed that the temperature changes from T1 to T2. At this time, as shown in a diagram on the right side of FIG. 2A, the objective lens 232 and the supporting mechanism therefor are deformed by a temperature change, whereby the focus position varies. Specifically, as shown in the graph on the left side of FIG. 2A, the focus position is Z1 at the temperature T1 and the focus position moves to Z2 when the temperature T2 is reached for the same focus adjustment amount V1. Note that the focus positions Z1, Z2 can be conceptually considered in the graph in this way. However, the focus position does not expressly appear in an OCT image and it is generally difficult to know where in the image the focus position is present.

If a relationship of the temperature T and the focus position Zf is expressed with the focus adjustment amount Vf fixed, the focus position Zf varies due to the temperature T even at the same focus adjustment amount Vf as shown in FIG. 2B. Here, the temperature T and the focus position Zf are expressed by a linear relationship. How the focus position Zf changes in relation to the temperature T depends on the structure of the objective optical system 23 and is not necessarily expressed by such a linear relationship. However, if a situation where the imaging apparatus 100 as in this embodiment is installed is considered, a temperature change range in the apparatus is at most about 10° C. Thus, an approximation to such a linear relationship is realistic if the temperature change is within such a range.

To keep the focus position Zf constant between a plurality of number of times of imaging performed at a time interval, the focus adjustment amount Vf needs not be made constant, but rather the focus adjustment amount needs to be changed to correspond to the temperature change. In an example shown in FIGS. 2A and 2B, a value of the focus adjustment amount Vf needs to be so set that the focus position Z1 at the temperature T1 and the focus adjustment amount V1 is kept also at the temperature T2. To enable this, how the focus adjustment amount Vf is changed to keep the focus position Zf constant against a temperature change needs to be clarified. A process for enabling this is described below.

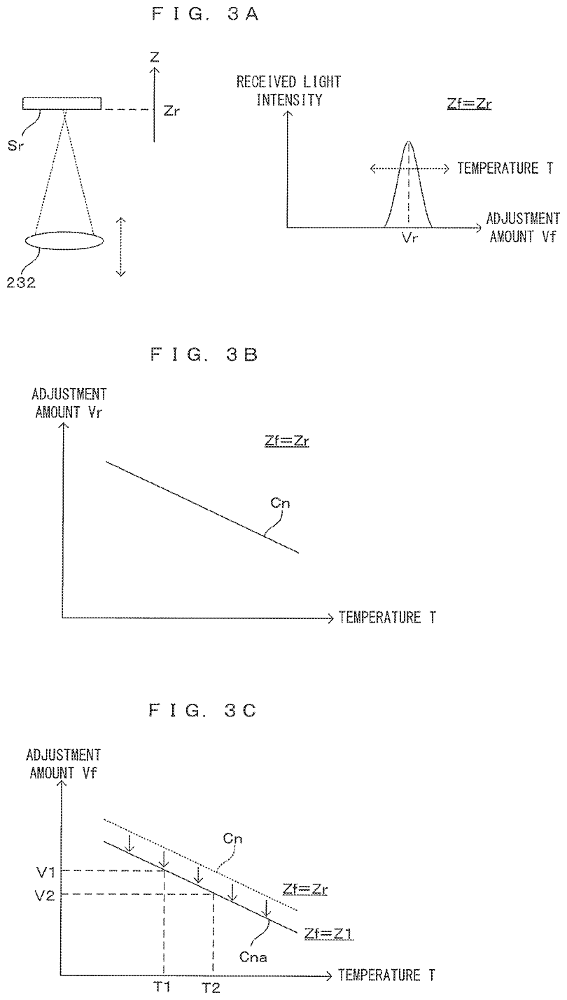

FIGS. 3A to 3C are principle diagrams showing how to obtain a focus adjustment amount corresponding to a temperature change. As shown in a diagram on the left side of FIG. 3A, an appropriate reflecting surface Sr is arranged on an object side light path where the well plate is arranged during imaging, and Zr denotes a Z-direction position of this reflecting surface Sr. Here, illumination light is caused to be incident on the reflecting surface Sr via the objective lens 232 and a received light intensity of reflected light from the reflecting surface Sr is detected. The intensity of the reflected light is thought to be maximum when the focus of the objective lens 232 is on the reflecting surface Sr.

Accordingly, as indicated by dotted-line arrows in the diagram on the left side of FIG. 3A, the received light intensity of the reflected light is detected while the focus position is changed by variously changing and setting the focus adjustment amount Vf. Then, as shown in a graph on the right side of FIG. 3A, the received light intensity is maximum when the focus adjustment amount Vf is a certain value Vr. At this time, the focus of the objective lens 232 is regarded to be on the reflecting surface Sr.

If the above measurement is conducted for various temperatures T, a curve Cn (straight line in this example) representing a relationship of the temperature T and the focus adjustment amount Vr at which the received light intensity is maximum is obtained as shown in FIG. 3B. This relationship represents a focus adjustment amount Vr necessary to maintain the focus position Zf at a constant value Zr regardless of the temperature.

To apply this relationship to the aforementioned example by assuming that this relationship is also valid at another focus position, the curve Cn may be moved in parallel to obtain a new curve Cna passing through a point (T1, V1) as shown in FIG. 3C. This curve Can corresponds to the one representing a correlation relationship of the temperature T and the focus adjustment amount Vf when the focus position Zf is fixed at Z1. Thus, a focus adjustment amount V2 corresponding to an arbitrary temperature T2 can be determined from the curve Cna.

Such a temperature characteristic possibly includes an individual difference of the apparatus. However, in the same individual apparatus, such a temperature characteristic does not largely vary with time. Accordingly, the above correlation relationship of the temperature and the focus adjustment amount may be obtained as a temperature characteristic of the apparatus at an appropriate timing (e.g. at the time of shipment or maintenance) for each individual imaging apparatus 100. In the following imaging operation, the focus position can be kept constant regardless of a temperature change using this relationship.

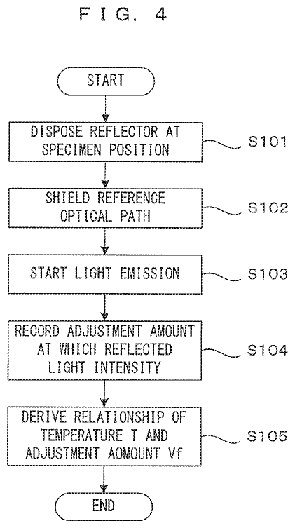

FIG. 4 is a flow chart showing a process of obtaining the correlation relationship of the temperature and the focus adjustment amount. To uniquely determine the focus position, a reflector serving as a dummy specimen is disposed at an appropriate position (Step S101). That position can be typically a position where the specimen is placed during imaging, i.e. a position corresponding to a lower surface position when the well plate WP is housed in the chamber 11. Further, since it is sufficient to obtain only an intensity of reflected light, the optical path of reference light is shielded at appropriate position (Step S102).

In this state, the emission of light from the light source 21 is started (Step S103), and the intensity of reflected light received by the photodetector 26 is detected while the objective lens 232 is moved. Then, a value of the focus adjustment amount Vf when the intensity is maximum is recorded. This is performed for various temperatures (Step S104).

From that result, the correlation relationship of the temperature T and the focus adjustment amount Vf to keep the focus position Zf constant can be specified as a temperature characteristic of the objective optical system 23 (Step S105).

A temperature characteristic in the reference optical system 24 can be basically obtained also by the same way of thinking. However, since the intensity of the reference light does not change even if the position of the reference mirror 243 is merely changed, the process of FIG. 4 cannot be directly applied. In this case, the following process may be performed.

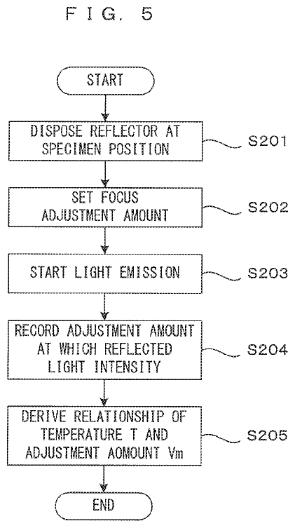

FIG. 5 is a flow chart showing a process of obtaining a correlation relationship of the temperature and the mirror adjustment amount. Here, the adjustment amount given from the CPU 301 to the mirror controller 313 to set the position of the reference mirror 243 is referred to as a “mirror adjustment amount” and denoted by Vm. In this process, a reflector is disposed at the position of the specimen during imaging similarly to the process for the objective optical system 23 (Step S201). The reference optical path is not shielded and, instead of this, the focus adjustment amount Vf is set at an appropriate value (Step S202). In this way, an objective optical path length is specified.

If the emission of light from the light source 21 is started in this state (Step S203), interference light is detected when the reference optical path length is equal to the objective optical path length. That is, the reference optical path length can be indirectly known via the fixed objective optical path length by detecting the intensity of the interference light. Accordingly, by changing the mirror adjustment amount Vm, the interference light received by the photodetector 26 is detected while the reference mirror 243 is moved. Then, a value of the mirror adjustment amount Vm when the objective optical path length and the reference optical path length become equal is recorded. The value of the mirror adjustment amount Vm when the objective optical path length and the reference optical path length become equal can be easily obtained by confirming a tomographic image generated from the interference light. Then, this is performed at various temperatures (Step S204). From that result, the correlation relationship of the temperature T and the mirror adjustment amount Vm to keep the reference optical path length constant can be specified as the temperature characteristic of the reference optical system 24 (Step S205).

If the temperature characteristics of the objective optical system 23 and the reference optical system 24 are obtained as described above, the imaging conditions (focus position and reference optical path length) in each imaging can be kept constant also in time lapse imaging in which an imaging time interval is long and a temperature change during the imaging time interval cannot be ignored.

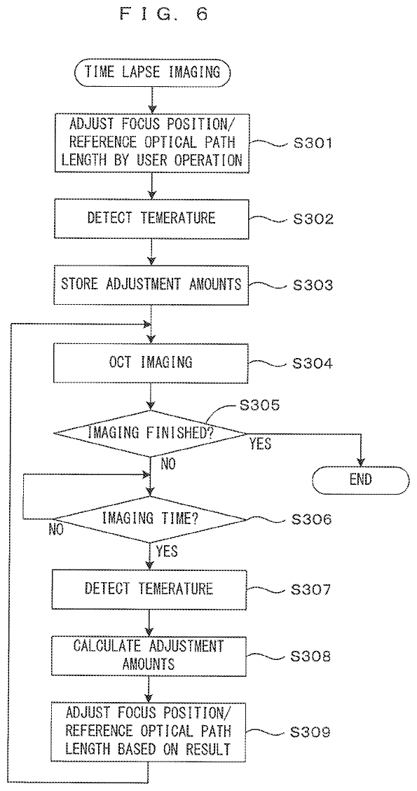

FIG. 6 is a flow chart showing time lapse imaging. It is assumed that a specimen to be imaged is cultured in the chamber 11 in advance. First, imaging conditions matching the specimen are set by a user operation to obtain an image corresponding to a user's purpose. These imaging conditions to be set include the focus position and the reference mirror position (Step S301).

Then, the temperatures of the objective optical system 23 and the reference optical system 24 at this point of time are detected (Step S302), and values of the set focus adjustment amount Vf and mirror adjustment amount Vm are stored in a memory 303 in association with the detected temperatures (Step S303). Note that the values of the focus adjustment amount Vf and the mirror adjustment amount Vm are associated with the detected temperatures. However, what is actually stored in the memory 303 is only the values of the focus adjustment amount Vf and the mirror adjustment amount Vm, and may not include temperature information. This point is described later.

OCT imaging is performed by the imaging unit 2 under the imaging conditions set by the user in this way (Step S304). “Imaging” mentioned here may be merely a process of obtaining data on an interference light intensity or may be a process including an image processing based on the data. At any rate, an image of the imaging object is fixed as data at this point of time.

If imaging has been finished to capture a necessary number of images (YES in Step S305), the imaging process is finished at this point of time. Unless otherwise (NO in Step S305), a standby state is set until the next imaging time arrives (Step S306) to realize an imaging interval determined in advance. During this time, various image processings may be performed. The specimen after imaging is kept in the chamber 11 and continues to be cultured in the constant culture environment.

When the imaging time arrives (YES in Step S306), the temperatures of the objective optical system 23 and the reference optical system 24 at that point of time are detected (Step S307). Then, the focus adjustment amount Vf and the mirror adjustment amount Vm applied in imaging this time are calculated based on those temperature detection results and the set values of the focus adjustment amount Vf and the mirror adjustment amount Vm in the first imaging stored in the memory 303 (Step S308). A specific calculation method is described later.

The calculated focus adjustment amount Vf and mirror adjustment amount Vm are given to the focus controller 312 and the mirror controller 313 from the CPU 301. By doing so, the focus position of the objective lens 232 and the position of the reference mirror 243 are adjusted (Step S309). In this way, even if there is a temperature change from the last imaging, the same conditions as those of the last imaging should be realized for the focus position and the reference mirror position. By performing OCT imaging anew in this state (Step S304) and repeating the above process until a necessary number of times of imaging is finished, aimed time lapse imaging is finished.

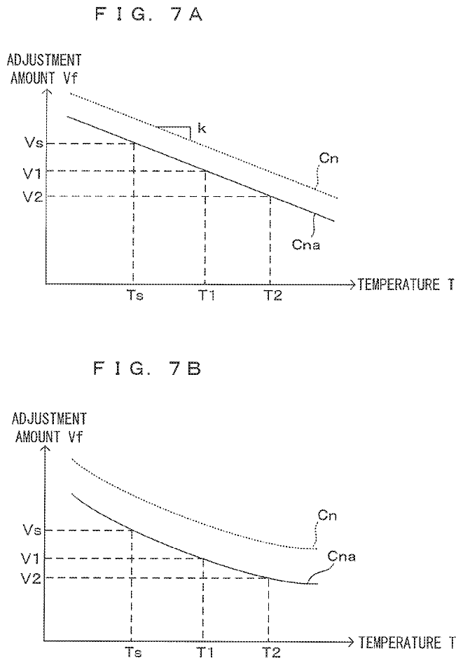

FIGS. 7A and 7B are graphs showing a method for obtaining the focus adjustment amount from the temperature characteristic of the objective optical system. Note that a method for obtaining the mirror adjustment amount Vm from the temperature characteristic of the reference optical system 24 is, in principle, the same. As shown in FIG. 7A, the temperature characteristic of the objective optical system 23 obtained in the process of FIG. 4 is generally specified only by the shape of the curve Cn (FIG. 3B), and the position of the curve in a vertical axis direction is not specified. In the first imaging in time lapse imaging, the value V1 of the focus adjustment amount set by the user and the temperature T1 at that time are determined, whereby the temperature characteristic curve Cna uniquely specified to include the position in the vertical axis direction is determined.

In the subsequent imaging, the focus adjustment amount V2 corresponding to the temperature T2 during imaging can be derived based on this curve Cna. By performing a focus adjustment corresponding to the focus adjustment amount V2, the focus position Zf should have the same value as the value Z1 during the first imaging.

As just described, to specify the specific temperature characteristic curve Cna corresponding to initial settings of the user from the generalized temperature characteristic curve Cn, two pieces of information, i.e. the value V1 of the focus adjustment amount and the temperature T1 at that time, are necessary. Here, if an appropriate reference temperature Ts is fixedly determined in advance as shown in FIG. 7A, it is no longer necessary to store temperature information. Specifically, only a value Vs which the curve Cna specified by the focus adjustment amount V1 and the temperature T1 at the time of the user setting takes at the reference temperature Ts may be stored as a value representing the focus adjustment amount at that time in the memory 303. By doing so, the curve Cna can be specified ex-post facto any time from the value Vs stored in the memory 303 and the value Vs determined in advance.

This method is equivalent to the specification of the curve Cna from two pieces of information V1, T1. As shown in FIG. 7B, the same way of thinking can be applied even if the temperature characteristic curve is represented by a more generalized curve.

Note that, if the temperature characteristic is represented by a straight line as shown in FIG. 7A, the generalized temperature characteristic can be more simply expressed only by a gradient k of the curve Cn obtained in advance. In this case, the focus adjustment amount V2 can be obtained as follows. As is clear from the relationship of FIG. 7A, the following equation:

Vs−V1=k(T1−Ts) (Equation 1)

is satisfied. The following equation:

Vs=V1+k(T1−Ts) (Equation 2)

is obtained by transposing (Equation 1). When the values V1, T1 are determined by user settings, the value Vs obtained by substituting these and known values k, Ts into (Equation 2) is stored in the memory 303.

For the focus adjustment amount V2 corresponding to the arbitrary temperature T2, the following equation:

V2=Vs−k(T2−Ts) (Equation 3)

is satisfied from FIG. 7A. Thus, the focus adjustment amount V2 can be directly obtained from the value Vs stored in the memory 303, the current temperature T2 and the known values k, Ts.

Note that if the curve Cn is shaped to be expressed by a second-order or higher polynomial as shown in FIG. 7B, the generalized temperature characteristic can be expressed by each term other than constant terms. When the constant terms are determined by values set by the user, the unique temperature characteristic curve Cna is specified.

As described above, in the imaging apparatus 100 of this embodiment, the correlation relationships of the adjustment amounts of the focus position and the reference mirror position and the temperature are obtained prior to imaging in realizing time lapse imaging by the OCT imaging apparatus. During imaging, the adjustment amounts of the focus position and the reference mirror position corresponding to the temperature at that point of time are derived using these correlation relationships. By doing so, variations of the focus position and the reference mirror position due to a temperature characteristic change between imaging operations are prevented. Since the imaging conditions are adjusted between the imaging operations, obtained images are suitably compared to each other.

As described above, in the above embodiment, the well plate WP corresponds to a “specimen container” of the invention. Further, the objective optical system 23, the reference optical system 24 and the CPU 301 respectively function as an “objective optical system”, a “reference optical system” and a “calculator” of the invention. Each of the temperature sensors 237, 247 functions as a “temperature detector” of the invention. Further, the focus controller 312 and the elevating mechanism 235 integrally function as a “focus adjustor” of the invention, whereas the mirror controller 313 and the advancing/retracting mechanism 245 integrally function as an “optical path adjustor” of the invention.

Note that the invention is not limited to the above embodiment and various changes other than the aforementioned ones can be made without departing from the gist of the invention. For example, in the above embodiment, each of the objective optical system 23 and the reference optical system 24 is provided with the temperature sensor, and the focus adjustment amount and the mirror adjustment amount are obtained based on the detection results of the temperature sensors. However, a sufficient effect is obtained even if the above configuration is applied only to either one of the objective optical system 23 and the reference optical system 24. Further, a structure of the apparatus may be configured to detect temperatures of an objective optical system and a reference optical system by a common temperature sensor.

Further, an imaging unit having the following configuration can be, for example, utilized as a modification of the above imaging unit 2. Specifically, it is known to use a beam splitter besides using an optical fiber coupler as described above in realizing a light demultiplexing/multiplexing function in an OCT device. Also in the case of using the imaging unit having such a configuration, the invention effectively functions. Further, the invention can also be applied to the following configuration.

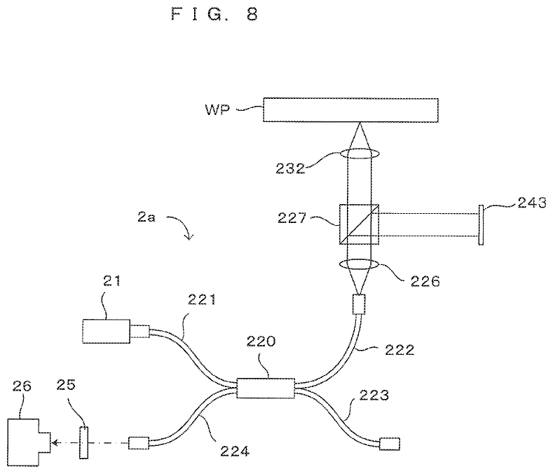

FIG. 8 is a diagram showing a modification of the imaging unit. In this example, an imaging unit 2a is provided with an optical fiber coupler 220. However, an optical fiber 223 is not used and a collimator lens 226 and a beam splitter 227 are provided for an optical path of light emitted from an optical fiber 222. An objective optical system 23 (objective lens 232) and a reference optical system 24 (reference mirror 243) are arranged in two optical paths branched by the beam splitter 227. In such a configuration, signal light and reference light are combined by the beam splitter 227, and interference light generated thereby is introduced to a photodetector 26 through the optical fibers 222, 224. The invention can also be suitably applied to an OCT device having such a configuration.

Further, although the above embodiment relates to an FD-OCT imaging apparatus, the invention can be applied to OCT imaging apparatuses in general regardless of the imaging principle. For example, the invention can be applied also to imaging apparatuses called TD-OCT (Time Domain OCT) imaging apparatuses and SD-OCT (Spectral Domain OCT) imaging apparatuses.

This invention is particularly effective for the purpose of regularly observing a specimen such as cells continuously cultured in a container and can be widely utilized in the fields of medicine and drug discovery.

Although the invention has been described with reference to specific embodiments, this description is not meant to be construed in a limiting sense. Various modifications of the disclosed embodiment, as well as other embodiments of the present invention, will become apparent to persons skilled in the art upon reference to the description of the invention. It is therefore contemplated that the appended claims will cover any such modifications or embodiments as fall within the true scope of the invention.

What is claimed is:

1. An imaging apparatus for optical coherence tomographic imaging, the imaging apparatus comprising:
an objective optical system which causes illumination light to be incident on an imaging object in a specimen container and receives reflected light from the imaging object;
a focus adjustor which adjusts a focus position of the objective optical system in an optical axis direction with respect to the specimen container;
a temperature detector which detects a temperature of the objective optical system; and
a calculator which calculates an adjustment amount of the focus position for an adjustment operation by the focus adjustor based on the temperature detected by the temperature detector and a correlation relationship of the temperature and the adjustment amount obtained in advance.

2. The imaging apparatus according to claim 1, wherein
the objective optical system includes an objective lens which converges the illumination light to the imaging object and condenses the reflected light, and
the focus adjustor adjusts a position of the objective lens in an optical path of the illumination light and the reflected light according to the adjustment amount.

3. The imaging apparatus according to claim 1, further comprising:
a reference optical system which forms a reference optical path of reference light for the reflected light; and
an optical path adjustor which adjusts an optical path length of the reference optical path, wherein
the temperature detector detects a temperature of the reference optical system and
the calculator calculates an adjustment amount of the optical path length for an adjustment operation by the optical path adjustor based on the temperature detected by the temperature detector and a correlation relationship of the temperature and the adjustment amount obtained in advance.

4. The imaging apparatus according to claim 3, wherein
the reference optical system includes a reference mirror and
the optical path adjustor adjusts a position of the reference mirror in the reference optical path according to the adjustment amount of the optical path length.

5. The imaging apparatus according to claim 1, further comprising an incubator which holds the specimen container in an internal space in which a temperature and a humidity are kept constant, wherein the imaging object to be imaged is in the specimen container held by the incubator.

6. An imaging apparatus for optical coherence tomographic imaging, the imaging apparatus comprising:
an objective optical system which causes illumination light to be incident on an imaging object in a specimen container and receives reflected light from the imaging object;
a reference optical system which forms a reference optical path of reference light for the reflected light;
an optical path adjustor which adjusts an optical path length of the reference optical path;
a temperature detector which detects a temperature of the reference optical system; and
a calculator which calculates an adjustment amount of the optical path length for an adjustment operation by the optical path adjustor based on the temperature detected by the temperature detector and a correlation relationship of the temperature and the adjustment amount obtained in advance.

7. The imaging apparatus according to claim 6, wherein
the reference optical system includes a reference mirror and
the optical path adjustor adjusts a position of the reference mirror in the reference optical path according to the adjustment amount of the optical path length.

8. The imaging apparatus according to claim 6, further comprising an incubator which holds the specimen container in an internal space in which a temperature and a humidity are kept constant, wherein the imaging object to be imaged is in the specimen container held by the incubator.

9. A control method for optical coherence tomographic imaging apparatus, the control method comprising:
detecting a temperature of an objective optical system which causes illumination light to be incident on an imaging object in a specimen container and receives reflected light from the imaging object;
calculating an adjustment amount of a focus position based on the temperature and a correlation relationship of the temperature and the adjustment amount obtained in advance; and
adjusting the focus position of the objective optical system in an optical axis direction with respect to the specimen container according to the adjustment amount.

10. A control method for optical coherence tomographic imaging apparatus, the control method comprising:
detecting a temperature of a reference optical system which forms an optical path of reference light;
calculating an adjustment amount of the optical path length of the reference light based on the temperature and a correlation relationship of the temperature and the adjustment amount obtained in advance; and
adjusting the optical path length according to the adjustment amount.

11. The control method according to claim 9, wherein a plurality of imaging is executed at predetermined time interval and detection of the temperature and adjustment of the adjustment amount are executed in each imaging.

12. The control method according to claim 10, wherein a plurality of imaging is executed at predetermined time interval and detection of the temperature and adjustment of the adjustment amount are executed in each imaging.
